(19) United States
(12) Patent Application Publication
(10) Pub. No.: US 2018/0085598 A1
(43) Pub. Date: Mar. 29, 2018

(54) Brachytherapy Applicator

(71) Applicant: Jiaju Zhang, Mount Sinai, NY (US)

(72) Inventor: Jiaju Zhang, Mount Sinai, NY (US)

(21) Appl. No.: 15/275,412

(22) Filed: Sep. 25, 2016

(51) Int. Cl. A61N 5/10

(52) U.S. Cl. CPC A61N 5/1016; A61N 2005/1008; A61N 5/1007

(57) ABSTRACT

A brachytherapy cervix applicator is disclosed comprising a cervix piece capable of reaching the cervix for radiation treatment and a uterus treatment piece comprising a moderately bendable and movable tube to aid insertion to uterus and an inflatable balloon in the uterus treatment piece to provide extra immobilization to the applicator inside the vaginal cavity. Optionally there is at least one radiation side-delivery channel located outside of the balloons.

### BACKGROUND OF THE INVENTION

### 1. Field of the Invention

The present invention is in the brachytherapy field of medical devices. Specifically, the present invention is an intra-cavitary, disposable, compact and CT/MRI compatible cervix applicator for high dose rate brachytherapy.

### 2. Description of the Prior Art

Brachytherapy is a specialized form of radiation therapy that entails the placement of an emitting radiation source (most commonly a radioactive isotope) in immediate proximity to macroscopic tumor and/or adjacent tissue at risk of harboring microscopic disease. It takes advantage of the inverse-square law, whereby radiation dose is inversely proportional to the square of the distance from the source. In practical terms, this allows for a very high dose to the tumor with relative sparing of the surrounding normal structures. Brachytherapy is the only demonstrated method of providing the high dose required to control cervical cancer (>80 Gray) without causing undue side effects. Brachytherapy requires the positioning specialized applicators that are specifically designed for each anatomic site or clinical circumstance into body cavities and tissues. In many ways, brachytherapy can be considered the ultimate form of conformal radiation therapy because it is unparalleled in its ability to direct a large dose of radiation to the tumor while minimizing exposure to surrounding sensitive normal structures.

Brachytherapy plays an important role in the treatment of cervical cancers. Brachytherapy for cervical or endometrial (uterine) cancers is typically done using a conventional applicator such as a “tandem and ovoid” (T&O) applicator or a “tandem and ring” (T&R) applicator, which reaches both the cervix and uterus. The tandem is a long, thin metal tube that passes through the cervix, into the uterus. The ovoids are circular hollow capsules and the ring is a hollow ring and they are placed in the vagina, pressed against the cervix. Once placed in the vagina, the applicator is connected to a machine that automatically feeds a radiation source into the applicator, where it remains for a predetermined time, known as the dwell time. Once the time is up, the machine removes the source and the applicator is removed from the vagina. The dwell time can be anywhere from 5-25 minutes.

The conventional cervix applicators, such as a Fletcher-style applicator (a “tandem and ovoid” applicator), is bulky and difficult to insert into the cervix. Frequently, the device has difficulty passing through the vagina because of its large size. Inserting such a device typically requires several physicians, including radiation oncologists, surgeons, and anesthesiologists to be present in the operating room. The challenges of using Fletcher-type devices are even greater when patients have abnormal anatomy. In addition, Fletcher-type of cervix applicators can also be complicated to assemble or disassemble for cleaning. Furthermore, it is well known that the Fletcher-type applicators are difficult to secure inside the vaginal cavity or uterus during treatment while a sufficiently secured applicator is critical for accurate delivery of radiation.

A more recent brachytherapy cervix applicator technology employs inflatable objects (generally call balloons). In this technology, the balloons (un-inflated) are inserted together with the radiation delivery tube into the cervix. The balloons are inflated once they are in the cervix. Examples are US2010/0145132, US2003/0153803, US2006/0173235, US2013/0085315, US2015/0065784, WO2014/43067, and WO2008/153715. The inflated balloons are meant to secure the radiation delivery tubes in places during treatment. However, the current balloon applicators employ at most two balloons and the balloons are used either in cervix or uterus separately but not used in cervix and uterus at the same time. Consequently it is difficult to secure the radiation delivery tubes during the treatment using the current balloon applicator technology. Furthermore, the current balloon applicators do not contain a flexible, soft and movable delivery tube that can reach the uterus. Another drawback of the current balloon applicators is that the radiation delivery pieces are only located inside the balloons.

### SUMMARY OF THE INVENTION

The brachytherapy applicator of the present invention comprises (1) one cervix piece capable of reaching the cervix for radiation treatment; (2) one uterus treatment piece capable of reaching the uterus comprising at least one balloon capable of providing extra support to the whole applicator, at least one moderately bendable and movable tube, at least one flexible delivery channel inside the at least one tube capable of accepting a stylet to move inside the delivery channel, at least one inflation channel with an air valve mechanism capable of controlling the inflation of the balloon to a desired size; (3) at least one supporting piece capable of holding the uterus treatment piece and cervix piece; and (4) at least one tube locking mechanism capable of controlling the length of the at least one tube in the uterus piece.

The present invention overcomes the drawbacks of the current balloon applicators by using a flexible, soft and movable tube and a balloon in the uterus piece to provide extra immobilization to the device inside the vaginal cavity in addition to the tubes and balloons used in cervix region in the current balloon applicator technologies. Optionally the applicator in the present invention comprises one or more side-delivery pieces capable of delivering the radiation closer to the targets.

### BRIEF DESCRIPTION OF THE DRAWINGS

FIG. 1 is an illustration of a delivery piece with a channel and a stylet.

FIG. 2 is an illustration of an inflation channel with an air valve mechanism.

FIG. 3 is an illustration of a uterus treatment piece.

FIG. 4 is an illustration of a cervix treatment piece.

FIG. 5 is an illustration of a cervix spacer piece.

FIG. 6 is an illustration of a supporting piece.

FIG. 7 is an illustration of an applicator of the present invention before insertion comprising a supporting piece of FIG. 6, a uterus treatment piece of FIG. 3, two cervix treatment pieces of FIG. 4, and two cervix spacer pieces of FIG. 5.

FIG. 8 is the applicator device of FIG. 7 when the piece is rotated 90°.

FIG. 9 is a prospective view of the applicator device of FIG. 7 after balloons have been inflated.

FIG. 10 is an illustration of the relative position of an applicator of FIG. 7 after insertion but before inflation.

FIG. 11 is an illustration of the relative position of the applicator device of FIG. 7 after inflation.

FIG. 12 is a prospective view of FIG. 10.

FIG. 13 is a prospective view of FIG. 11.

FIG. 14 is an illustration of another supporting piece.

FIG. 15 is the supporting piece of FIG. 14 when the piece is rotated 90°.

FIG. 16 is an illustration of another delivery piece with a channel with an anchor and a stylet.

FIG. 17 is an illustration of an applicator before insertion comprising a supporting piece of FIG. 14, a uterus treatment piece of FIG. 3, and a cervix piece comprising one balloon, one inflation channel with an air valve mechanism, and two side-delivery pieces of FIG. 16.

FIG. 18 is the applicator of FIG. 17 after the two balloons are inflated.

FIG. 19 is an illustration of anther uterus treatment piece comprising a uterus treatment piece of FIG. 3, and two additional side-delivery pieces.

FIG. 20 is the applicator of FIG. 19 with the balloon inflated.

### DETAILED DESCRIPTION OF THE INVENTION

The present invention is a compact balloon applicator, which improves upon currently available balloon applicators by virtue of its narrow design, easy insertion, and extra stabilization for use in high dose rate cervix cancer brachytherapy. The brachytherapy applicator of the present invention comprises (1) one cervix piece capable of reaching the cervix for radiation treatment; (2) one uterus treatment piece capable of reaching the uterus comprising at least one balloon capable of providing extra support to the whole applicator, at least one moderately bendable and movable tube, at least one flexible delivery channel inside the at least one tube and the delivery channel is capable of accepting a stylet capable of moving inside the at least one delivery channel, at least one inflation channel with an air valve mechanism capable of controlling the inflation of the balloon to a desired size; (3) at least one supporting piece capable of supporting the uterus treatment piece and cervix piece; and (4) at least one tube locking mechanism capable of controlling the length of the at least one tube in the uterus piece. Optionally the balloon applicator of the present invention comprises side-delivery pieces.

Throughout the specification, all pieces with a same number perform the same functions. For example, “3” is a stylet and it can be used in a uterus piece or a cervix piece. The letter “U” after a number means the piece is for uterus use while the letter “C” after a number means the piece is for cervix use. For example, “3U” means the stylet belongs to a uterus piece. A number after the letter “U” or “C” means the nth piece. For example, 3C2 means the second stylet in the cervix piece. When there is no number after the letter “U” or “C”, it means there is only one such a piece in the uterus piece or the cervix piece. Therefore, “3U” means a stylet in the uterus piece and there is only one stylet in the uterus piece.

Throughout the specification, a fluid means a gas (e.g., air) or a liquid (e.g., water or saline).

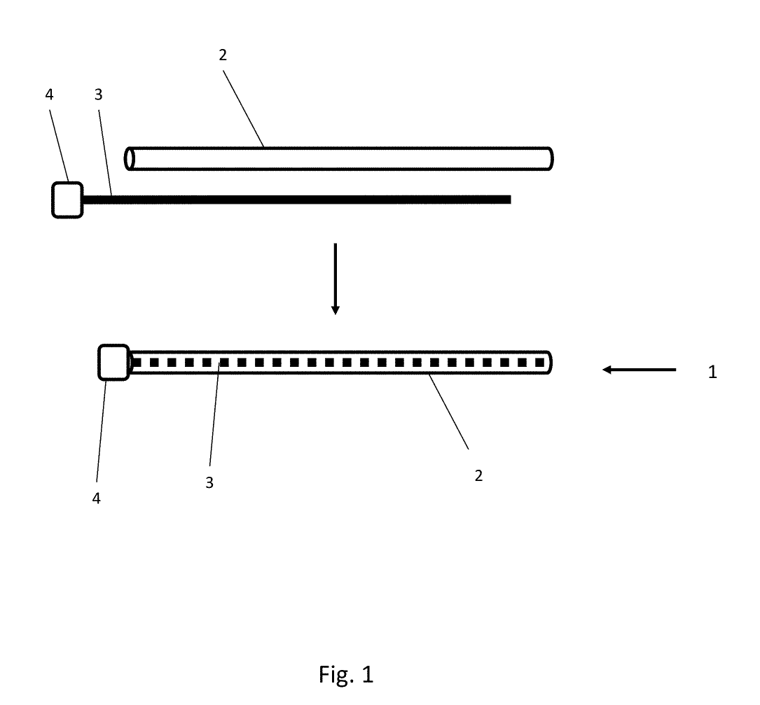

Referring to the invention in more detail, FIG. 1 illustrates a delivery piece (1) comprising a cylindrical, flexible delivery channel (2) and a stylet (3) with a stylet handle (4). The delivery piece (1) can be used in a uterus piece or a cervix piece. The delivery channel (2) is open at one end and close at the other. The stylet (3) is a long and thin wire and it is capable of inserting into the delivery channel (2) through the open end. There are three types of stylets, non-bendable (or rigid), moderately bendable, and easily bendable (or very flexible). The stylet (3) is able to slide back and forth inside the delivery channel (2) and its tip is able to reach the end of the delivery channel (2). The non-bendable stylet and the moderately bendable stylet are mean to facilitate the insertion of the applicator into uterus. The easily bendable stylet (3), without the stylet handle (4), is mean to carry a radiation source at its tip and meant to be inserted by a radiation source machine to deliver the radiation to targets.

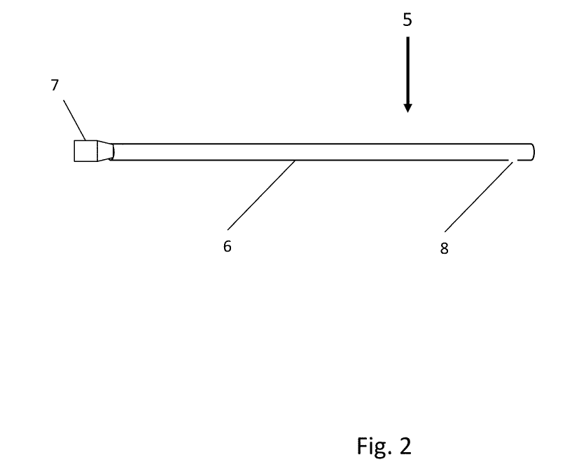

FIG. 2 is an illustration of an inflation piece (5) comprising an inflation channel (6) with an air valve mechanism (7) and an opening (8). The air valve mechanism (7) is a device capable of regulating the inflation of a balloon to a desired size. The valve is a one way valve and it is able to open and close depending on the air pressure and air direction provided by the pump. When inflating, the air comes from the air valve mechanism (7) and goes out through the opening (8) to the balloon. When deflating, the air valve mechanism (7) sucks out the air from the balloon. The inflation piece (5) always pairs up with a balloon and the unit is necessarily made air tight.

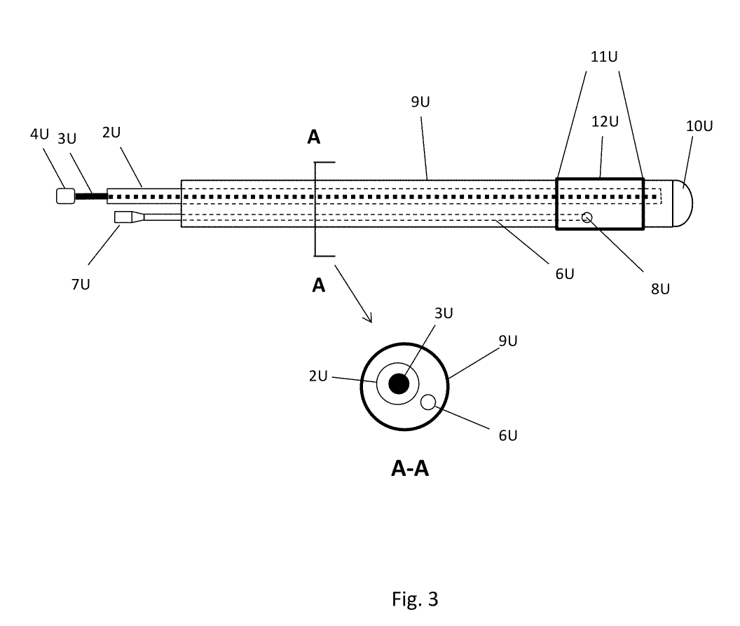

FIG. 3 is an illustration of a flexible uterus piece, also called a uterus treatment piece, with an additional cross sectional view. The uterus piece comprises a hollow tube (9U). The tube (9U) is a flexible and moderately bendable tube. A delivery piece (1) with a delivery channel (2U), a stylet (3U), and a stylet handle (4U) and an inflation piece (5) with an inflation channel (6U), an air valve mechanism (7U), and an opening (8U) are inserted into the tube (9U). Guide head (10U) is a soft and smooth piece capable of leading the tube (9U) during insertion. The use of guide head (10U) is to minimize potential injures to the patient during insertion of the applicator. A balloon (12U) is also shown closed to the end of the tube (9U) with its two ends sealed or tied at position (11U). When inflating, the fluid comes from the air valve mechanism (7U), moves along the inflation channel (6U) and goes out through the opening (8U) to the balloon (12U). When deflating, the air valve mechanism (7U) sucks out the fluid from the balloon (12U). The inflation channel (6U) can be a permanently built-in piece in the tube (9U), a standalone piece, or a partly built-in and a partly standalone piece.

The tube (9U) is sufficiently long (about 25 to 35 cm typically). The diameters of the tube (9U), the delivery channel (2U), and the inflation channel (6U) are typically about 4.7 mm (outer diameter), 2 mm (inner diameter), and 0.5 mm (inner diameter), respectively.

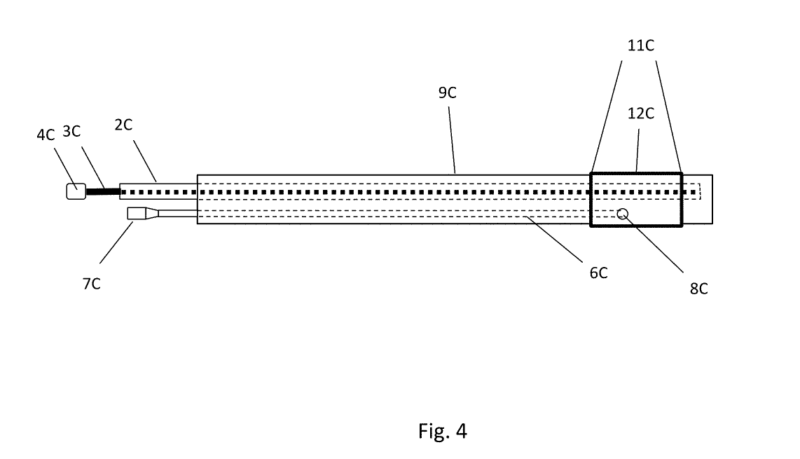

FIG. 4 is an illustration of a cervix treatment piece. The cervix treatment piece is similar to a uterus treatment piece. The only difference is that the cervix treatment piece does not need a guide head (10). A flexible tube (9C), a delivery piece (1) with a delivery channel (2C), a stylet (3C), and a stylet handle (4C), and an inflation piece (5) with an inflation channel (6C), an air valve mechanism (7C), and an opening (8C) are inserted into the tube (9C). A balloon (12C) is also shown with its two ends sealed or tied at position (11C).

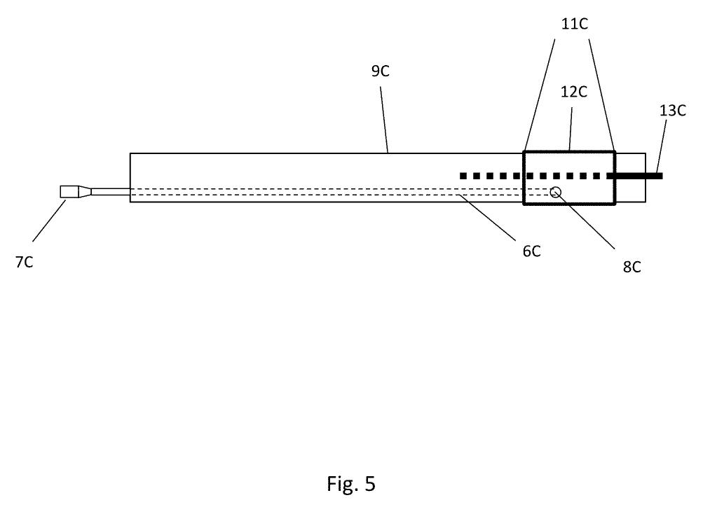

FIG. 5 is an illustration of a cervix spacer piece. The cervix spacer piece is the same as a cervix treatment piece except that, instead of a delivery piece (1), it contains a stabilizing pin (13C) inserted through the end of tube (9C). The main function of a cervix spacer piece is to push away the bladder or rectum to minimize the risk of radiation reaching the bladder and rectum. The current art is to use a cotton ball or a soft material to push away the bladder or rectum. The use of a balloon in the cervix spacer piece makes the job much easier. The other function of the cervix spacer piece is to stabilize the applicator.

Throughout the specification of the present invention, a cervix piece may comprise one or more cervix treatment piece, and optionally with one or more cervix spacer piece.

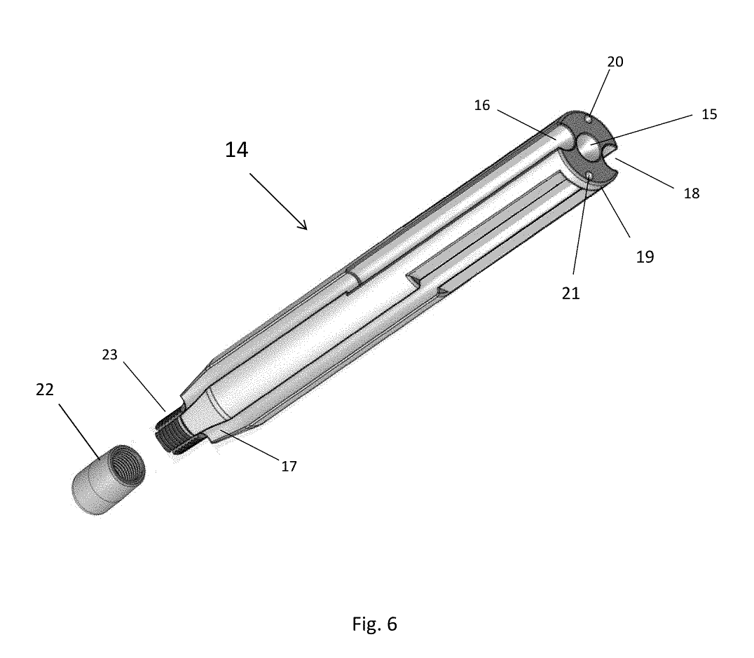

FIG. 6 is an illustration of a supporting piece (14) capable of holding the tube (9U) and tube (9C) together. Supporting piece (14) is a cylindrical object with cutouts and with a center hole (15) passing through the whole supporting piece. Tube (9U) of a uterus treatment piece of FIG. 3 is able to move or slide through the hole (15). A tube locking mechanism comprising an open cap (female) (22) with threads and a hollow cylinder with cutouts and threads (male) (23) is able to lock the tube (9U) to desired lengths. The hollow cylinder (23) is an extended part of the main supporting piece (14). After insertion of the tube (9U) into the hole (15) to a desired length, the cap (22) can be mated to the hollow cylinder (23). Turning the cap (22) is able to squeeze the hollow cylinder (23) and tighten the tube (9U). Four troves (16, 17, 18, and one hidden in the opposite side of 17) are meant to host tubes (9C) for cervix piece. Also shown are two small holes (20 and 21) in top piece (19) at the ends of trove (17) and the hidden trove.

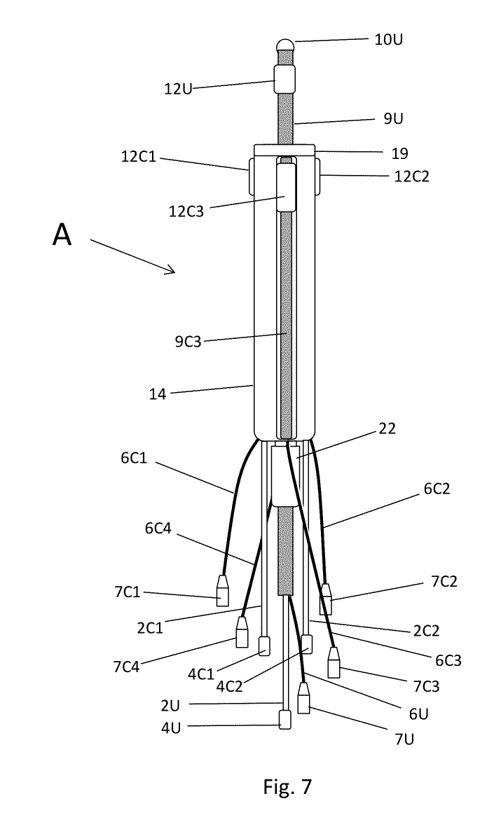

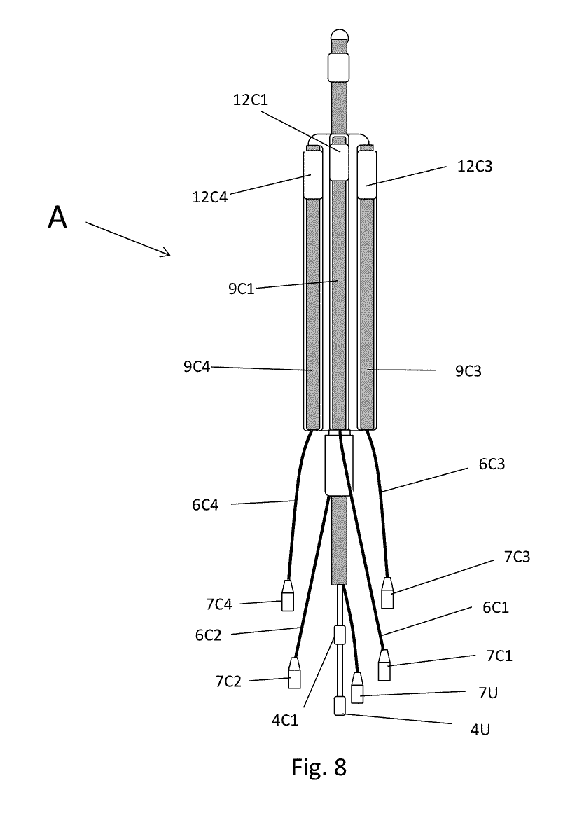

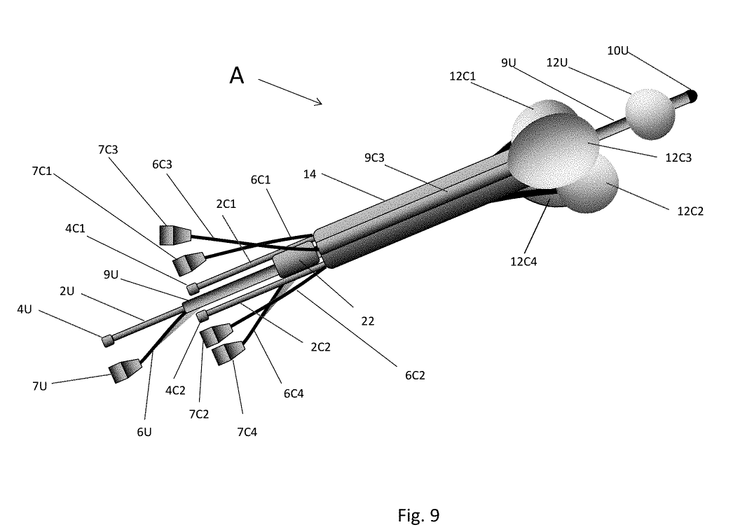

FIG. 7, FIG. 8, and FIG. 9 are illustrations of the same applicator “A” of the present invention but in different view. FIG. 7 is an illustration of an applicator before insertion comprising a supporting piece (14) of FIG. 6, a uterus treatment piece of FIG. 3, and a cervix piece comprising two cervix treatment pieces of FIG. 4 and two cervix spacer pieces of FIG. 5. FIG. 8 is the applicator device of FIG. 7 when the piece is rotated 90°. FIG. 9 is a prospective view of the applicator device of FIG. 7 after balloons have been inflated. The uterus treatment piece of FIG. 3 is inserted into the supporting piece (14) through hole (15). The parts belonging to the uterus treatment piece are a balloon (12U), an inflation channel (6U) with an air valve mechanism (7U), and a delivery channel (2U) with a stylet with a handle (4U). The first cervix treatment piece of FIG. 4 is in trove (16) of the supporting piece (14). The parts belonging to the first cervix treatment piece are a balloon (12C1), an inflation channel (6C1) with an air valve mechanism (7C1), and a delivery channel (2C1) with a stylet with a handle (4C1). The second cervix treatment piece of FIG. 4 is in trove (18) of the supporting piece (14). The parts belonging to the second cervix treatment piece are a balloon (12C2), an inflation channel (6C2) with an air valve mechanism (7C2), and a delivery channel (2C2) with a stylet with a handle (4C2). The first cervix spacer piece of FIG. 5 is in trove (17) with its stabilizing pin (13C) inserting into the hole (20). The parts belonging to the first cervix spacer piece are a balloon (12C3) and an inflation channel (6C3) with an air valve mechanism (7C3). The second cervix spacer piece of FIG. 5 is in the hidden trove with its stabilizing pin (13C) inserting into the hole (21). The parts belonging to the second cervix spacer piece are a balloon (12C4) and an inflation channel (6C4) with an air valve mechanism (7C4). The balloon (12U) in the uterus treatment piece plays a crucial role of proving additional support to the balloon applicator “A”. Without the balloon (12U), the applicator is difficult to secure at desired position.

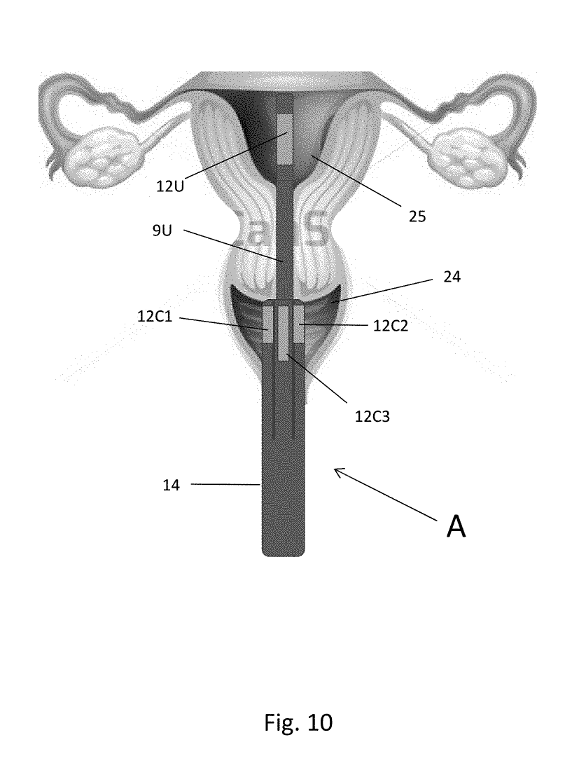

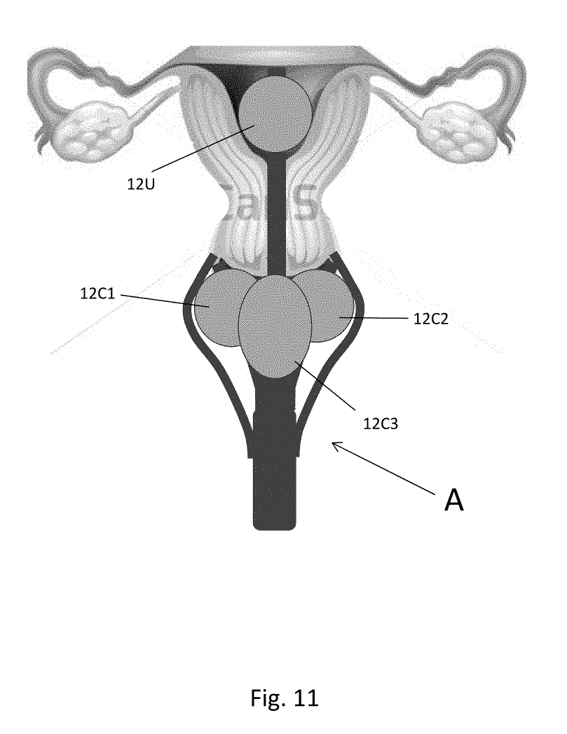

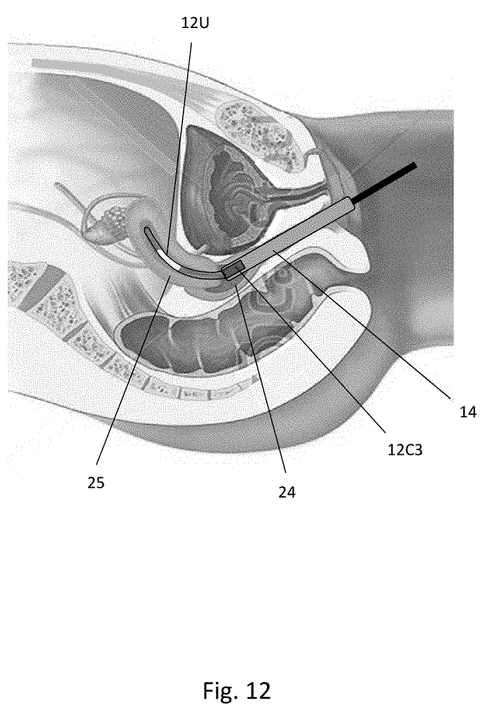

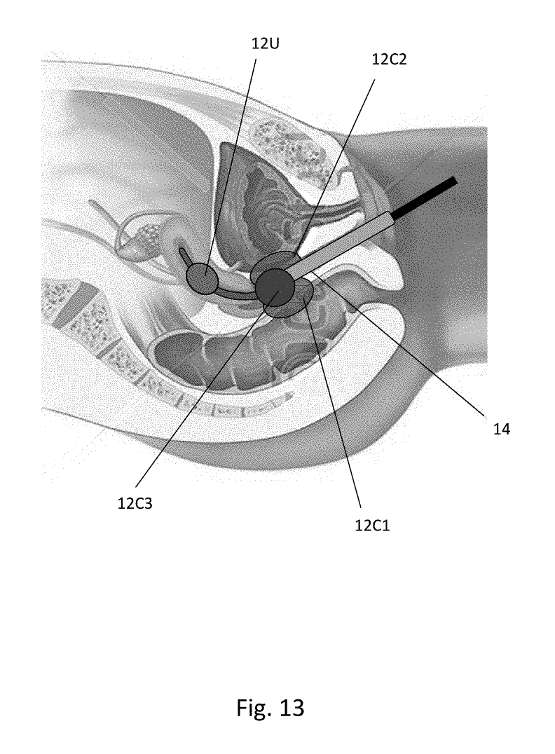

FIG. 10 to FIG. 13 are the same illustrations but in different views showing the relative positions of the applicator of FIG. 7 inside a cervix and a uterus before and after the inflation of the balloons. They are mainly for clarification purpose. Labels 24 and 25 in FIG. 10 and FIG. 12 are vagina and uterus, respectively. As shown in FIG. 12, the angle between the cervix and the uterus is quite large and the flexible uterus piece has been inserted into the uterus. Without the use of the flexible uterus piece of the present invention, it would be very difficult or cumbersome for a physician to insert an applicator into the uterus. As shown in FIG. 13, the balloon (12C3) in the cervix spacer piece pushes away the bladder while the balloon (12C4) pushes away the rectum.

The components of the applicators can be made of different materials with different mechanical properties. In particular, the tube material can be hard, soft, or even both hard and soft at the same time at different location, and it can bend at any angle without breaking off.

The degree of difficulty of insertion of the applicator into the uterus depends on the geometry of the vaginal cavity. Insertion of the tubes into the cervix is relatively easy while insertion of a tube into the deeper uterus is more difficult. The geometry of a vaginal cavity varies with patients and with the size of tumors. When the tumor is relatively small, the insertion is relatively easy and sometime the uterus tube (9U) and other cervix tubes (9C) are hard enough so that there is no need to use a stylet to aid insertion. When the tumor is of a moderate size and the vaginal cavity is not too small, the insertion of the tubes can be aided with a moderately bendable stylet. However, when the tumor is large, the vaginal cavity is small and the angle between the cervix and the uterus may be as large as 90°. In this case, the strong, a non-bendable stylet is first used to facilitate the insertion of the tube (9U) into the turning section between the cervix and the uterus. After the tube reaches the turning section, the non-bendable stylet is withdrawn from the delivery channel. The moderately bendable stylet at this time can be inserted into the delivery channel and the tube can then be inserted into the uterus with the aid of the moderately bendable stylet. After the tube has reached the uterus, the moderately bendable stylet is withdrawn and the bendable stylet with radiation source can then be inserted. The design of the tube in the present invention allows insertion of the applicator into the uterus with different angles and different size of vaginal openings. This is the key discovery of the present invention.

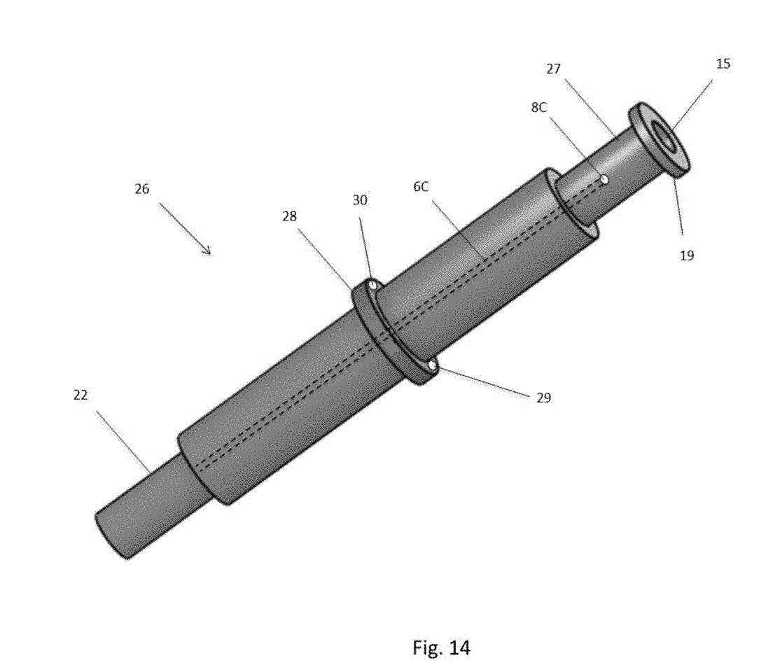

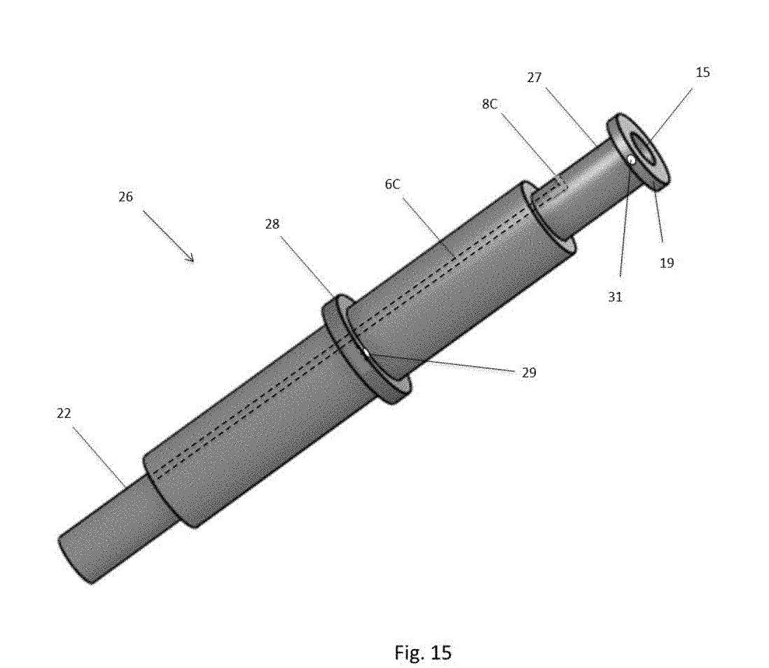

FIG. 14 is an illustration of another supporting piece (26) of the present invention. FIG. 15 is the same as FIG. 14 when the piece is rotated 90°. The center hole (15) and the tube locking mechanism (22 and 23) in the supporting piece (26) are similar to those in supporting piece (14) in FIG. 6. The main body of the supporting piece (26) is a hollow cylinder, i.e., a cylinder with a center hole (15) running all the way to the end of the tube locking mechanism (22 and 23).

Ring (28) is a tightly fitted piece around the main body of the supporting piece (26) and the ring (28) is preferably movable along the main body of the supporting piece (26). The ring (28) has two holes (29 and 30) through which the delivery channels are able to pass freely. Neck (27) is where a balloon is attached. Top piece (19) has two small holes (31 and the hidden hole opposite of 31) capable of accepting delivery channels. Also shown are an inflation channel 6C and an opening 8C.

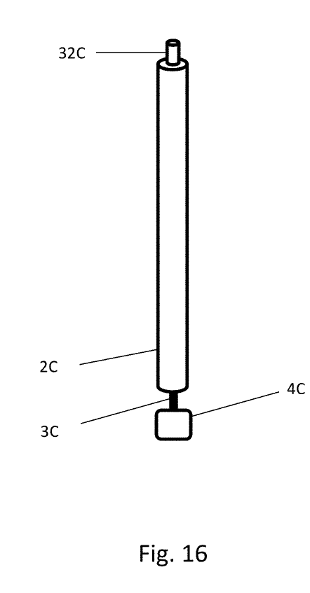

FIG. 16 is an illustration of another delivery piece with a channel (2C), an anchor (32C) at the end of the channel (2C), and a stylet (3C) and a stylet handle (4C).

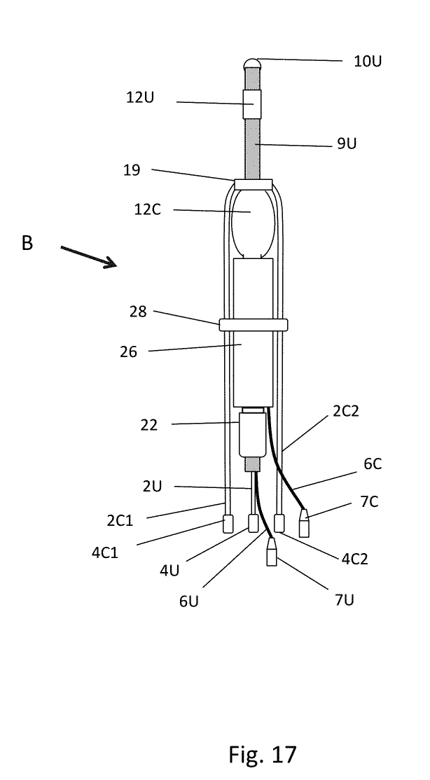

FIG. 17 is an illustration of an applicator “B” before insertion comprising a supporting piece of FIG. 14, a uterus treatment piece of FIG. 3, and a cervix treatment piece comprising one balloon (12C), one inflation channel (6C) with an air valve mechanism (7C), and two side-delivery channels (2C1 and 2C2) of FIG. 16 located at the outside of the balloon (12C). Also included in FIG. 17 are two stylets with handles (4C1 and 4C2). The two delivery channels (2C1 and 2C2) pass through the two holes (29 and 30) in the ring (28) of FIG. 14 and insert using the two anchors (32C) of FIG. 16 into the two holes (31 and the hidden hole opposite of 31) in the top piece (19) of FIG. 15. To inflate the balloon (12C), a fluid is pushed out by the air valve mechanism (7C), moves along the inflation channel (6C) through the supporting piece (26) of FIG. 14, and exits the opening (8C) of the supporting piece of FIG. 14.

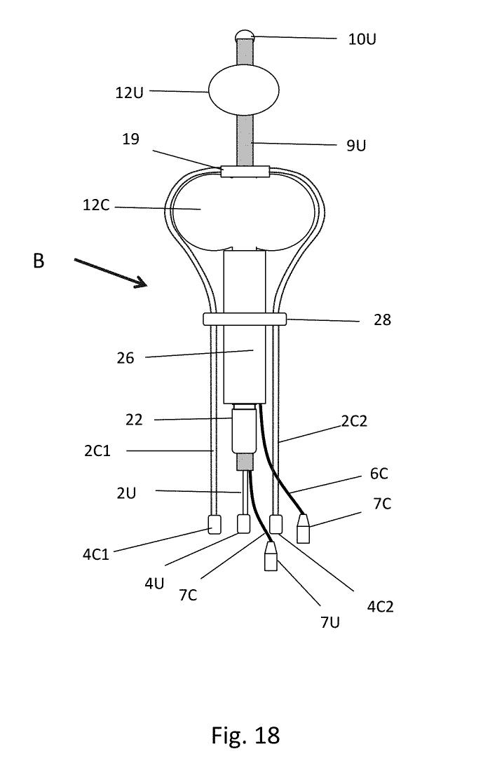

FIG. 18 is the applicator of FIG. 17 after the two balloons (12U and 12C) are inflated. The inflated balloon can have an oval shape, a round shape, or a shape similar to the contour of the space between cervix, bladder, and rectum after the balloon is inflated. The side-delivery channels (2C1 and 2C2) can approach the targets more closely than the current arts where the delivery channels are always located inside the balloons. Although only two side-delivery channels are shown in this example, it should be understood by a skilled in the art that, in another embodiment, up to 12 side-delivery channels (2C1 to 2C12) may also be used with slight modifications to the numbers of holes in top piece (19) and ring (28).

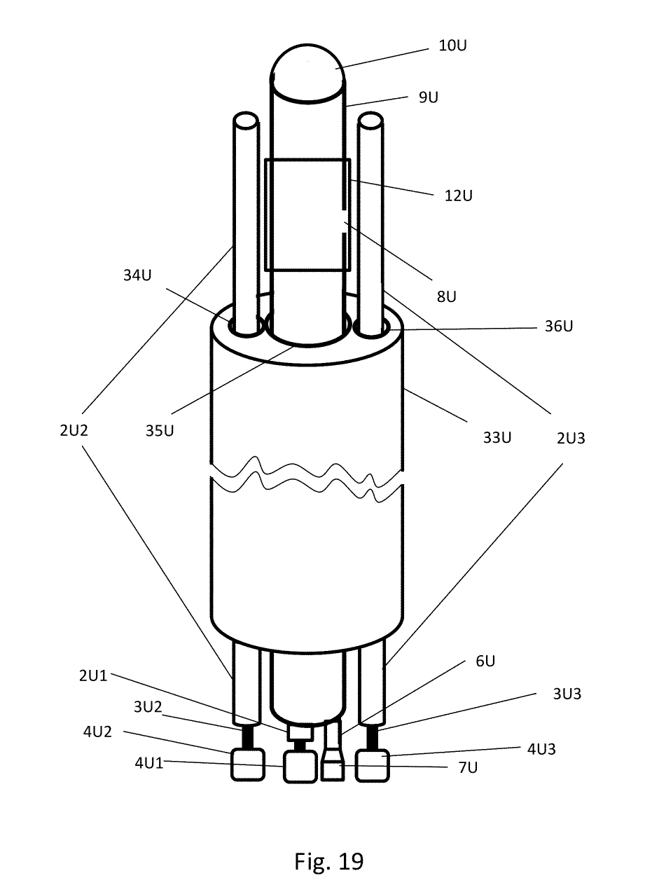

FIG. 19 is an illustration of anther uterus treatment piece with two side-delivery pieces comprising a uterus treatment piece of FIG. 3, two additional side-delivery channels (2U2 and 2U3) located outside of the balloon (12U), and an outer tube (33U) with a top piece with three holes (34U, 35U and 36U). The pieces belonging to the uterus treatment piece are a tube (9U) with a guide head (10U), a balloon (12U), a delivery channel (2U1), a stylet with a handle (4U1), and an inflation channel (6U) with an air valve mechanism (7U) and an opening (8U). The flexible delivery channel (2U2) passes through outer tube (33U) through hole (34U). The uterus piece of FIG. 3 passes through outer tube (33U) through hole 35U. The flexible delivery channel (2U3) passes through outer tube (33U) through hole 36U.

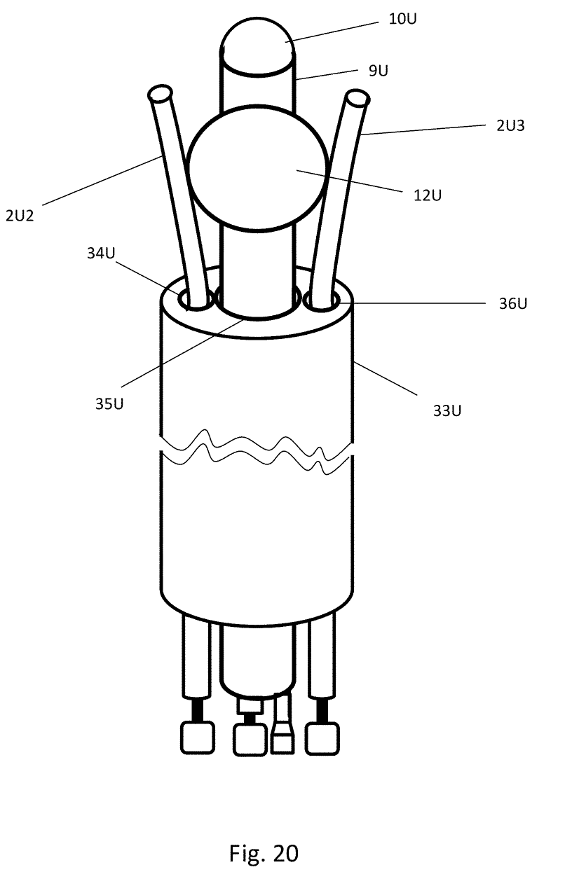

FIG. 20 is the applicator of FIG. 19 with the balloon inflated. The side-delivery channels (2U2 and 2U3) can approach the targets more closely than the current arts. Although only two side-delivery channels are shown in this example, it should be understood by a skilled in the art that, in another embodiment, with simple modifications, more than two side-delivery pieces can be used.

In all examples before insertion, the balloons are not inflated. Following insertion, the balloons are inflated to desired sizes. Typically the inflated volume of a balloon is about 5-20 ml. The balloons can be inflated with same source of a fluid. However, it is preferred to inflate each balloon separately so that the size of each balloon can be controlled.

As various changes could be made to the above examples without departing from the scope of the invention, it is intended that all matters contained in the above description and shown in the accompanying drawings shall be interpreted as illustrative and not in a limiting sense. All drawings are not in scale.

I claim:

1. A brachytherapy balloon applicator comprising (1) a cervix piece capable of reaching the cervix for radiation treatment; (2) a uterus treatment piece capable of reaching the uterus comprising at least one moderately bendable and movable tube capable of bending at various angles, at least one balloon capable of providing extra support to the whole applicator when inflated, at least one inflation channel with an air valve mechanism capable of controlling the inflation of the at least balloon to a desired size, at least one flexible delivery channel capable of passing through the at least one moderately bendable and movable tube along the interior of the moderately bendable and movable tube, at least one stylet capable of inserting into the at least one delivery channel; (3) at least one supporting piece capable of supporting the uterus piece and the cervix treatment piece; and (4) at least one tube locking mechanism capable of controlling the length of the at least one moderately bendable and movable tube in the uterus treatment piece.

2. The brachytherapy applicator of claim 1 wherein the uterus treatment piece comprises one moderately bendable and movable tube, one balloon, one inflation channel with an air valve mechanism, one flexible delivery channel, one guide head, wherein the inflation channel and the flexible delivery channel pass through the moderately bendable and movable tube, wherein the supporting piece comprises a center hole through which the moderately bendable and movable tube is able to pass, and wherein the tube locking mechanism comprises an open cap tightening mechanism.

3. The brachytherapy applicator of claim 2 wherein the cervix piece comprises one balloon and one inflation channel with an air valve mechanism capable of controlling the inflation of the balloon to a desired size.

4. The brachytherapy applicator of claim 3 wherein the cervix piece comprises two flexible side-delivery pieces with an anchor on each capable of adopting a similar contour to the outside surface of the one balloon in the cervix piece when inflated; wherein the one balloon in the cervix piece has an oval shape, a round shape, or a shape similar to the contour of the space between cervix, bladder, and rectum after the one balloon in the cervix piece is inflated; wherein the supporting piece additionally comprises a movable ring with two holes around the main body of the supporting piece, a top piece with two holes, an inflation channel along the center hole of the supporting piece wherein the inflation channel has an opening to the balloon, and a neck capable of hosting the balloon; and wherein the two flexible side-delivery pieces pass through the two holes on the ring and anchor at the two holes on the top piece.

5. The brachytherapy applicator of claim 4 wherein the cervix piece comprises up to twelve flexible side-delivery channel located outside the balloon, wherein the ring and the top piece have up to twelve holes.

6. The brachytherapy applicator of claim 2 wherein the cervix piece comprises two cervix treatment pieces and two cervix spacer pieces; wherein each cervix treatment piece comprises a flexible tube, a flexible delivery piece, a balloon, an inflation channel with an opening to the balloon, and an air valve mechanism capable of controlling the inflation of the balloon to a desired size; wherein each cervix spacer piece comprises a flexible tube, a balloon, an inflation channel with an opening to the balloon, an air valve mechanism capable of controlling the inflation of the balloon to a desired size, and a stabilizing pin inserted through one end of the tube; and wherein the supporting piece further comprises four troves capable of hosting the two cervix treatment pieces and the two cervix spacer pieces and a top piece with two holes wherein the two stabilizing pins are able to insert into the two holes in the top piece.

7. The brachytherapy applicator of claim 2 wherein the uterus treatment piece further comprises two or more flexible side-delivery pieces located outside the one balloon, an outer tube with a top piece with a center hole and two or more side holes; wherein the uterus piece of claim 2 is able to pass through the outer tube through the center hole, the two or more flexible side-delivery pieces are able to pass through the outer tube through the two or more side holes; and wherein the one balloon is able to push and bend the two or more flexible side-delivery pieces.

8. The brachytherapy applicator of claim 7 wherein the uterus treatment piece comprises two flexible side-delivery pieces on the outside of the one balloon.

9. The brachytherapy applicator of claim 7 wherein the cervix piece comprises one balloon and one inflation channel with an air valve mechanism capable of controlling the inflation of the balloon to a desired size, two flexible side-delivery pieces with an anchor on each capable of adopting a similar contour to the outside surface of the one balloon when the one balloon in the cervix piece is inflated; wherein the one balloon in the cervix piece has an oval shape, a round shape, or a shape similar to the contour of the space between cervix, bladder, and rectum after the one balloon is inflated; wherein the supporting piece additionally comprises a movable ring with two holes around the main body of the supporting piece, a top piece with two holes, an inflation channel along the center hole of the supporting piece wherein the inflation channel has an opening to the balloon, and a neck capable of hosting the balloon; and wherein the two flexible delivery pieces pass through the two holes on the ring and anchor at the two holes on the top piece.

10. The brachytherapy applicator of claim 9 wherein the cervix piece comprises up to twelve flexible side-delivery pieces located outside the balloon and wherein the ring and the top piece have up to twelve holes.

11. The brachytherapy applicator of claim 7 wherein the cervix piece comprises two cervix treatment pieces and two cervix spacer pieces, wherein each cervix treatment piece comprises a flexible tube, a flexible delivery piece, a balloon, an inflation channel with an opening to the balloon, and an air valve mechanism capable of controlling the inflation of the balloon to a desired size; wherein each cervix spacer piece comprises a flexible tube, a balloon, an inflation channel with an opening to the balloon, an air valve mechanism capable of controlling the inflation of the balloon to a desired size, and a stabilizing pin inserted through one end of the tube; and wherein the supporting piece further comprises four troves capable of hosting the two cervix treatment pieces and the two cervix spacer pieces and a top piece with two holes wherein the two stabilizing pins are able to insert into the two holes in the top piece.
